(19) United States
(12) Patent Application Publication
(10) Pub. No.: US 2016/0220150 A1
(43) Pub. Date: Aug. 4, 2016

(54) SURGICAL INSTRUMENT WITH MAGNETIC SENSOR

(71) Applicant: COVIDIEN LP, Mansfield, MA (US)

(72) Inventor: Alexey Sharonov, Bethany, CT (US)

(21) Appl. No.: 14/917,667

(22) PCT Filed: Aug. 13, 2014
(86) PCT No.: PCT/US2014/050825

§ 371 Date: Mar. 9, 2016

Related U.S. Application Data
(60) Provisional application No. 61/882,323, filed on Sep. 25, 2013.

(51) Int. Cl. A61B 5/107; A61B 17/10; A61B 5/05; A61B 17/068

(52) U.S. Cl. CPC A61B 5/1076; A61B 17/068; A61B 17/105; A61B 5/05; A61B 2017/00039

(57) ABSTRACT

A surgical instrument includes an end effector, a magnetic field sensor assembly, and a processor. The end effector includes first and second tissue contacting surfaces configured to receive tissue therebetween. The first tissue contacting surface is movable relative to the second tissue contacting surface between a spaced apart position and an approximated position. The magnetic field sensor assembly includes a first magnetic field sensor disposed on the first tissue contacting surface and a first magnet disposed on the second tissue contacting surface. The processor is connected to the magnetic field sensor. The processor determines a distance between the first and second tissue contacting surfaces based on a detectable signal received from the first magnetic field sensor.

### CROSS-REFERENCE TO RELATED APPLICATIONS

This application is a U.S. National Stage Application filed under 35 U.S.C. §371(a) of International Patent Application No. PCT/US2014/050825, filed Aug. 13, 2014, which claims benefit of, and priority to, U.S. Provisional Patent Application 61/882,323, filed on Sep. 25, 2013. The entire contents of each of the above applications is hereby incorporated by reference.

### BACKGROUND

1. Technical Field

The present disclosure relates to a surgical instrument, and more particularly, to a surgical instrument including a magnetic field sensor assembly for determining tissue thickness.

2. Background of Related Art

Various surgical procedures are performed in a minimally invasive manner. This includes forming a small opening through a body wall of a patient, e.g., in the abdomen, and inserting surgical instruments therethrough to perform surgical procedures. Due to the relatively small interior dimensions of the access devices used in endoscopic procedures, only elongated, small-diametered instrumentation may be used to access the internal body cavities and organs. Typically, such instruments are limited in their ability to sense and/or control conditions and/or parameters during an operation, such as, for example, the thickness of tissue positioned between tissue contacting surfaces of an end effector of the surgical instrument.

Accordingly, a need exists for surgical instruments that can sense the amount of tissue positioned between tissue contacting surfaces of an end effector of the surgical instrument and provide this information to the user prior to operation of the surgical instrument.

### SUMMARY

In accordance with an embodiment of the present disclosure, there is provided a surgical instrument including an end effector, a magnetic field sensor assembly, and a processor. The end effector includes first and second tissue contacting surfaces configured to receive tissue therebetween. The first tissue contacting surface is movable relative to the second tissue contacting surface between a spaced apart position and an approximated position. The magnetic field sensor assembly includes a first magnetic field sensor disposed on the first tissue contacting surface and a first magnet disposed on the second tissue contacting surface. Alternatively, the first magnetic field sensor may be disposed on the second tissue contacting surface and the first magnet may be disposed on the first tissue contacting surface. The processor is connected to the first magnetic field sensor. The processor determines a distance between the first and second tissue contacting surfaces based on a detectable signal received from the first magnetic field sensor.

In an embodiment, the surgical instrument may further include a contact sensor disposed on the first tissue contacting surface. The contact sensor may monitor contact between tissue and the first tissue contacting surface during approximation of the first tissue contacting surface toward the second tissue contacting surface.

In another embodiment, the first tissue contacting surface may be pivotably coupled with the second tissue contacting surface about a pivot. In particular, the first magnetic field sensor may be disposed adjacent the pivot. The magnetic field sensor assembly may further include a second magnetic field sensor disposed distal of the first magnetic field sensor and a second magnet disposed distal of the first magnet, such that during approximation of the first tissue contacting surface toward the second tissue contacting surface, the first magnetic field sensor contacts tissue while the second magnetic field sensor is spaced apart from tissue.

In an embodiment, the first magnet and the first magnetic field sensor may be in a superposed relation in the approximated position. The first magnetic field sensor may be a Hall effect sensor. Alternatively, the first magnetic field sensor may include a magnetoresistive film.

In accordance with another aspect of the present disclosure, there is provided a method of determining tissue thickness. The method includes placing tissue between a first tissue contacting surface and a second tissue contacting surface of an end effector of a surgical instrument; approximating the first and second tissue contacting surfaces; generating a detectable signal; and calculating a distance between the first and second tissue contacting surfaces based on the detectable signal. The detectable signal is generated by a magnetic field sensor on the first tissue contacting surface in response to a magnetic field of a magnet on the second tissue contacting surface.

In an embodiment, the method may further include determining an initial contact between tissue and the first tissue contacting surface. Furthermore, generating a detectable signal may include generating the detectable signal at the time of initial contact between tissue and the first tissue contacting surface.

In accordance with another embodiment of the present disclosure, there is provided a method of determining tissue thickness. The method includes placing a magnet on a first side of tissue; placing a magnetic field sensor mounted on a surgical instrument on a second side of tissue; generating a detectable signal; and calculating a distance between the magnet and the magnetic field sensor based on the detectable signal. The second side is opposite of the first side. The detectable signal is generated by the magnetic field sensor in response to a magnetic field of the magnet.

### DESCRIPTION OF THE DRAWINGS

Various embodiments of the present disclosure are described hereinbelow with reference to the drawings, wherein:

FIG. 1 is a perspective view of a surgical instrument in accordance with an embodiment of the present disclosure;

FIG. 2 is a side, cross-sectional view of a main body of the surgical instrument of FIG. 1, shown in a first, unapproximated condition;

FIG. 3 is an enlarged, side cross-sectional view of a tool assembly of the surgical instrument of FIGS. 1 and 2, shown in the first, unapproximated condition;

FIG. 4 is an enlarged, side cross-sectional view of the tool assembly of the surgical instrument of FIGS. 1 and 2, shown in a second, approximated condition;

FIG. 5 in an enlarged, side cross-sectional view of the tool assembly of the surgical instrument of FIGS. 1 and 2, shown after completion of a firing stroke;

FIG. 6 is a perspective view of a surgical instrument in accordance with another embodiment of the present disclosure;

FIG. 7 is a top perspective view of a handle assembly of the surgical instrument of FIG. 6 with a portion of a handle section removed therefrom;

FIG. 8 is a side cross-sectional view of the distal end of the surgical instrument of FIGS. 6 and 7, shown in a first condition;

FIG. 9 is a side cross-sectional view of the distal end of the surgical instrument of FIGS. 6 and 7, shown in a second condition;

FIG. 10 is a perspective view of a surgical instrument in accordance with another embodiment of the present disclosure;

FIG. 11 is a side, cross-sectional view of the surgical instrument of FIG. 10; and

FIG. 12 is a partial perspective view of a magnet assembly for use with the surgical instrument of FIG. 10.

### DETAILED DESCRIPTION

Embodiments of the present disclosure will now be described in detail with reference to the drawings, in which like reference numerals designate identical or corresponding elements in each of the several views. As used herein, the term “distal,” as is conventional, will refer to that portion of the instrument, apparatus, device or component thereof which is farther from the user while, the term “proximal,” will refer to that portion of the instrument, apparatus, device or component thereof which is closer to the user. In the following description, well-known functions or constructions are not described in detail to avoid obscuring the present disclosure in unnecessary detail.

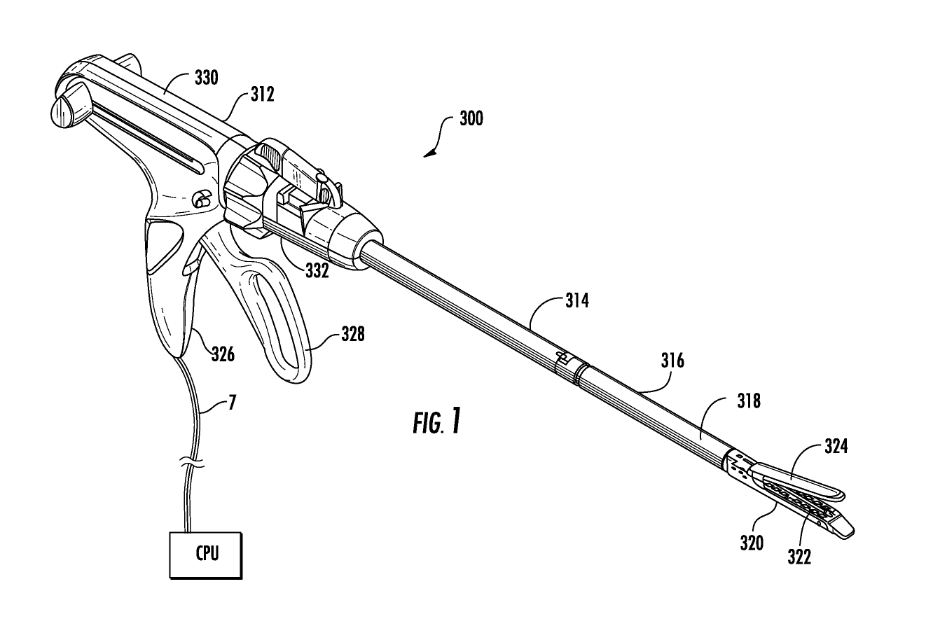

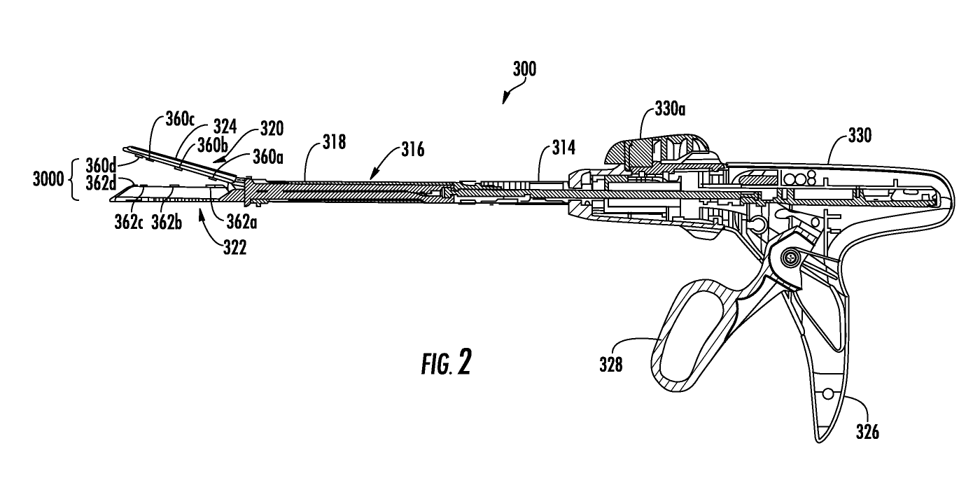

With reference now to FIGS. 1 and 2, there is illustrated a surgical instrument 300 including a magnetic field sensor assembly 3000 (FIG. 2) in accordance with an embodiment of the present disclosure. Surgical instrument 300 includes a handle assembly 312 and an elongated body 314. Handle assembly 312 includes a stationary handle member 326, a movable handle or trigger 328 and a barrel portion 330. A disposable loading unit or DLU 316 is releasably secured to a distal end of elongated body 314. DLU 316 includes a proximal body portion 318, which forms an extension of elongated body 314, and a distal tool assembly or end effector 320 including a cartridge assembly 322 and an anvil assembly 324. Tool assembly 320 is pivotably connected to body portion 318 about an axis substantially perpendicular to the longitudinal axis of elongated body 314. Reference may be made to U.S. Pat. No. 8,281,937, the entire contents of which are incorporated herein by reference, for a more detailed discussion of the structure and operation of surgical instrument 300.

With particular reference now to FIGS. 2-4, surgical instrument 300 includes a magnetic field sensor assembly 3000 disposed in tool assembly 320. Magnetic field sensor assembly 3000 includes a plurality of magnets 362a, 362b, 362c, 362d disposed on tissue contacting surface 322a (FIG. 3) of cartridge assembly 322 and a plurality of magnetic field sensors 360a, 360b, 360c, 360d disposed on tissue contacting surface 324a (FIG. 3) of anvil assembly 324. Magnets 362a, 362b, 362c, 362d may be permanent magnets or electromagnets.

Magnetic field sensors 360a, 360b, 360c, 360d may be any type of sensor capable of generating a detectable signal in response to the presence of a magnetic field. In embodiments, the magnitude of the detectable signal generated by the sensor varies with the strength of the magnetic field detected. Suitable magnetic field sensors include, e.g., Hall effect sensors. As those skilled in the art will appreciate, a Hall effect sensor is a transducer that varies its output voltage (the detectable signal) in response to a magnetic field. Magnetoresistive films may be used in making the magnetic field sensor. For example, a magnetic field sensor made from thin film giant magnetoresistive (GMR) materials may be placed adjacent a source for producing a magnetic field. In embodiments, the GMR material and the source for producing the magnetic field may be placed on respective tissue contacting surfaces 322a, 324a of surgical instrument 300. Accordingly, the distance from the GMR material to the source for producing the magnetic field would vary with changes in the thickness of tissue. The distance from the GMR material to the source for producing the magnetic field may be calculated based on the magnitude of the detectable signal generated by the GMR material based on the strength of the magnetic field at any given time.

Magnetic field sensors 360a, 360b, 360c, 360d are pre-calibrated for magnets 362a, 362b, 362c, 362d. For any particular magnet 362a, 362b, 362c, 362d and orientation of sensor 360a, 360b, 360c, 360d with respect to that magnet, distance between sensor 360a, 360b, 360c, 360d and the respective magnets 362a, 362b, 362c, 362d can be determined by means of interpolation of pre-calibrated values. A sensor reading proportional to the magnetic field is transformed to a distance measurement by means of interpolation or lookup table in which each value of the magnetic field measurement is converted to the thickness of tissue.

Magnetic permeability of the material is given by the equation

μ=μ₀(1+χₘ) (Eq. 1)

where μ₀ is permeability of free space and χₘ is a magnetic susceptibility of material. For diamagnetic and paramagnetic materials, magnetic susceptibility is extremely small χₘ<<1) (e.g., χₘ of water is −9.035×10⁻⁶). Human tissue and other nonferrous and ferrimagnetic materials do not differ substantially from that of free space in terms of magnetic field propagation. As such, the permeabilities of diamagnetic and paramagnetic materials do not differ substantially from that of free space and these materials being inserted between magnet and magnetometer substantially have no effect on distance measurements.

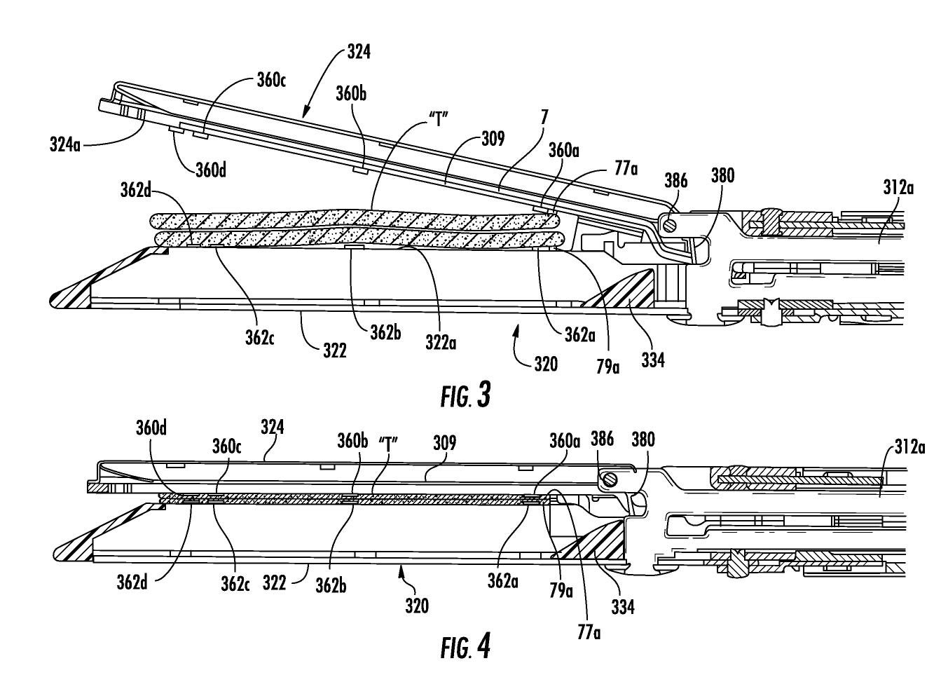

With particular reference now to FIGS. 3 and 4, magnets 362a, 362b, 362c, 362d and corresponding magnetic field sensors 360a, 360b, 360c, 360d are positioned on respective tissue contacting surfaces 322a, 324a, such that a magnet 362a, 362b, 362c, 362d and a corresponding magnetic field sensor 360a, 360b, 360c, 360d form a pair and are in a superposed relation when anvil assembly 324 is in the approximated position (FIG. 4) to clamp tissue “T” between tissue contacting surfaces 322a, 324a.

Sensors 360a, 360b, 360c, 360d may be selectively connected to a processor or a central processing unit (CPU) (FIG. 1) for monitoring, controlling, processing and/or storing information observed, measured, sensed and/or transmitted from any of the elements of components of the surgical instruments prior, during and/or after the surgical procedure. Sensors 360a, 360b, 360c, 360d may be electrically connected via a wire 7 (FIG. 3) or connected wirelessly to CPU. The data collected by sensors 360a, 360b, 360c, 360d are sent to CPU. The data are transformed to a distance measurement by means of interpolation in which each value of magnetic field measurement is converted to a tissue thickness. The tissue thickness may be displayed on an indicator (not shown) in units of length (thickness) or, alternatively, graphically represented for potential use of the device in any particular case, e.g., whether the device is appropriately sized for the procedure. It is contemplated that the display may be the monitor to which images are shown from the camera used during laparoscopic surgery. It is also contemplated that the display may be on the instrument itself, for example, on barrel portion 330 of surgical instrument 300, or any other portion of surgical instrument 300 that is easily viewed by the user during surgery.

Tool assembly 320 may further include contact sensors 77a, 79a connected to CPU to detect an initial contact between tissue “T” and tissue contacting surface 324a of anvil assembly 324. For example, contact sensors 77a, 79a may include pressure sensors, electrical contacts and sensing circuits, force transducers, piezoelectric elements, piezoresistive elements, metal film strain gauges, semiconductor strain gauges, inductive pressure sensors, capacitive pressure sensors, and potentiometric pressure transducers.

Contact sensors 77a, 79a may be disposed adjacent sensor 360a and magnet 362a, respectively. In particular, contact sensors 77a detect an initial contact between tissue contacting surface 324a and tissue “T” during approximation of anvil assembly 324. In this manner, magnetic field sensor 360a can measure tissue thickness when tissue “T” is initially brought into contact with tissue contacting surface 324a of anvil assembly 324, which, in turn, enables the surgeon to measure the substantially uncompressed thickness of tissue “T”. As surgical instrument 300 is being clamped onto tissue “T”, contact sensors 77a, 79a may provide the user with an indication (e.g., audio, visual, tactile, etc.) as to when tissue “T” is initially brought into contact with tissue contacting surface 324a of anvil assembly 324.

In use, with cartridge assembly 322 and anvil assembly 324 in spaced relation to one another, target tissue “T” is placed therebetween. With the target tissue “T” positioned between cartridge assembly 322 and anvil assembly 324, anvil assembly 324 is approximated toward cartridge assembly 322. Contact sensor 77a, 79a may detect the initial contact between tissue “T” and tissue contacting surface 324a. At this time, magnetic field sensor 360a may measure the magnetic field and send the data to CPU, which determines the substantially uncompressed thickness of tissue “T”. The tissue thickness in the uncompressed state is measured and/or recorded. Thereafter, cartridge assembly 322 and anvil assembly 324 are further approximated until all sensors 360a, 360b, 360c, 360d are in a superposed relation with the respective magnets 362a, 362b, 362c, 362d. Then, the tissue thickness in the compressed state is measured and/or recorded.

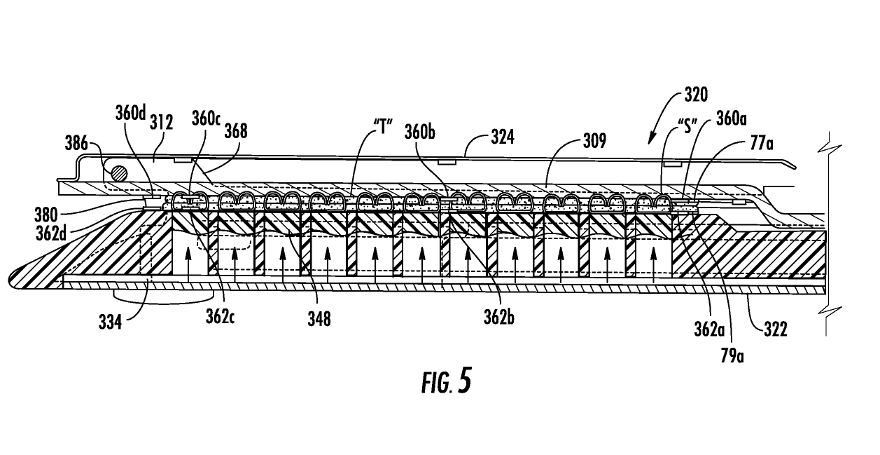

With reference to FIGS. 1 and 5, anvil assembly 324 is movable in relation to cartridge assembly 322 between an open position (FIG. 3) spaced from cartridge assembly 322 and an approximated or clamped position (FIG. 4) in juxtaposed alignment with cartridge assembly 322. To approximate cartridge and anvil assemblies 322 and 324, movable handle 328 is moved toward stationary handle 326, through an actuation stroke. Subsequent movement of movable handle 328 through the actuation stroke effects advancement of an actuation shaft and a firing rod (not shown). As the actuation shaft is advanced, the firing rod is also advanced.

The firing rod is connected at its distal end to axial drive assembly 312a (FIG. 4) such that advancement of the firing rod effects advancement of drive assembly 312a. As drive assembly 312a is advanced, cam roller 386 moves into engagement with cam surface 309 of anvil assembly 324 to urge anvil assembly 324 toward cartridge assembly 322, thereby approximating cartridge and anvil assemblies 322 and 324 and clamping tissue “T” therebetween.

To fire surgical instrument 300, movable handle 328 is moved through a second actuation stroke to further advance the actuation shaft and the firing rod distally. As the firing rod is advanced distally, drive assembly 312a (FIG. 4) is advanced distally to advance actuation sled 334 through staple cartridge assembly 322 to simultaneously sever tissue “T” with knife 380 and drive pushers 348 to sequentially eject staples “S” from cartridge assembly 322.

Surgical instrument 300 is adapted to receive DLU's having staple cartridges with staples in linear rows having a length of from about 30 mm to about 60 mm. For example, each actuation stroke of movable handle 328 during firing of surgical instrument 300 may advance the actuation shaft approximately 15 mm, although other lengths are envisioned. Accordingly, in embodiments to fire a cartridge assembly having a 45 mm row of staples, movable handle 328 must be moved through three actuation strokes after the approximating or clamping stroke of movable handle 328.

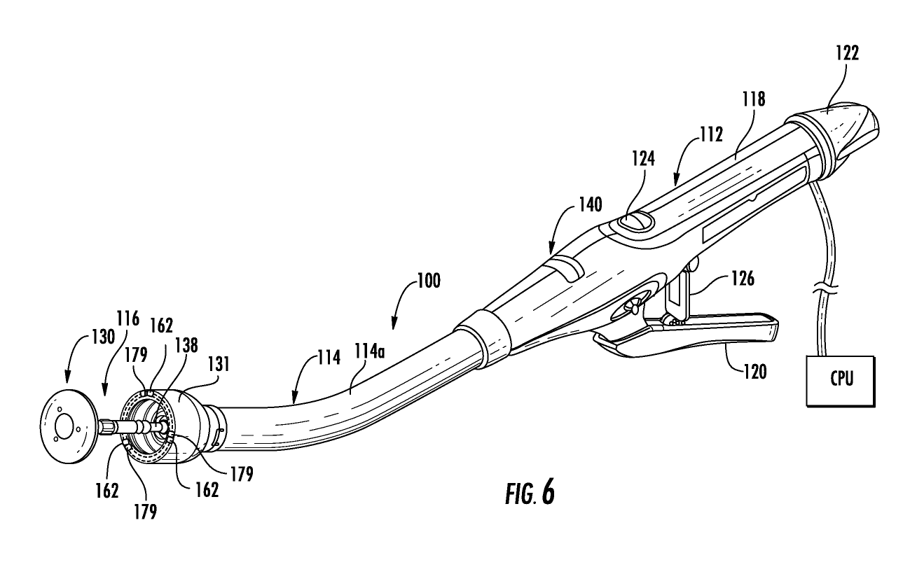

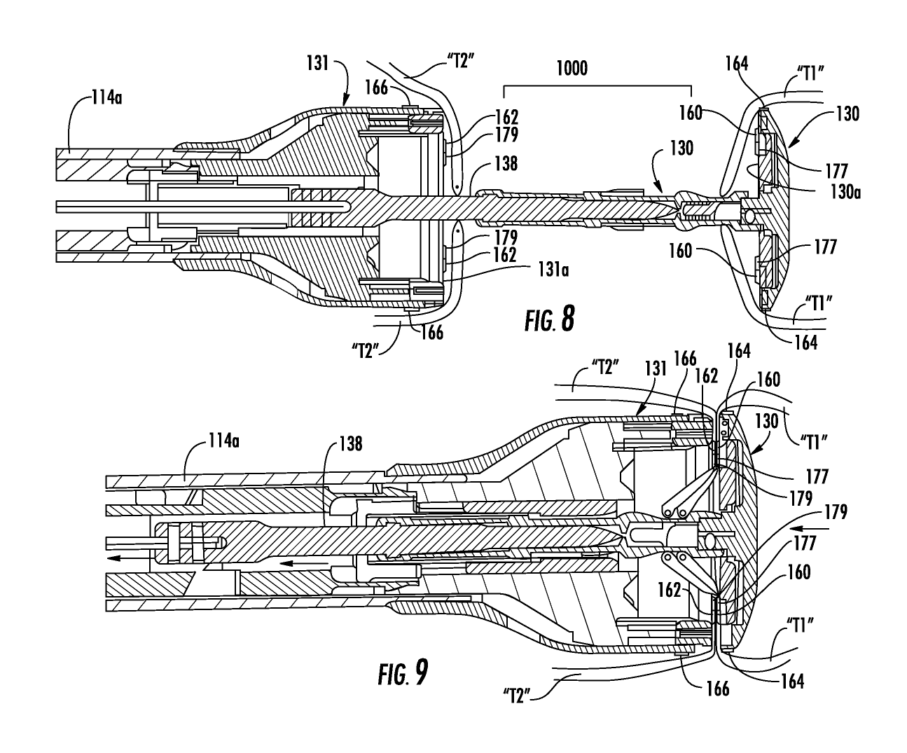

With reference now to FIGS. 6 through 9, a surgical instrument including a magnetic field sensor assembly 1000 (FIG. 8) in accordance with another embodiment of the present disclosure is generally designated as 100. Surgical instrument 100 includes a proximal handle assembly 112, an elongated central body portion 114 including a curved elongated outer tube 114a, and a distal head portion 116. Alternately, in some surgical procedures, e.g., the treatment of hemorrhoids, it is desirable to have a substantially straight, preferably shortened, central body portion. The length, shape and/or the diameter of body portion 114 and head portion 116 may also be tailored to a particular surgical procedure being performed.

With continued reference to FIG. 6, handle assembly 112 includes a stationary handle 118, a firing trigger 120, a rotatable approximation knob 122 and an indicator 124. Head portion 116 includes an anvil assembly 130 and a shell assembly 131. Reference may be made to U.S. Pat. No. 7,802,712, the entire contents of which are incorporated herein by reference, for a more detailed discussion of the structure and operation of surgical instrument 100.

With additional reference to FIGS. 6, 8, and 9, magnetic field sensor assembly 1000 includes a plurality of magnets 162 disposed on tissue contacting surface 131a of shell assembly 131 and a plurality of magnetic field sensors 160 disposed on tissue contacting surface 130a of anvil assembly 130. Magnets 162 and magnetic field sensors 160 may be constructed as described above in connection with the embodiments of FIGS. 1 to 5.

With continued reference to FIGS. 8 and 9, magnets 162 and corresponding magnetic field sensors 160 are positioned on respective tissue contacting surfaces 130a, 131a such that a magnet 162 and a corresponding magnetic field sensor 160 form a pair and are in a superposed relation when anvil assembly 130 is in the approximated position (FIG. 9) to clamp tissue “T₁”, “T₂” between tissue contacting surfaces 130a, 131a. The magnetic field reading or detectable signal from magnetic field sensors 160 is sent to a processor (CPU) (FIG. 6). The data are transformed to a distance measurement by means of interpolation in which the value of the magnetic field measurement is translated to a tissue thickness. The tissue thickness can be displayed in any suitable manner, such as, for example, on indicator 124 (FIG. 6) in units of length (thickness) or, alternatively, graphically represented for potential use of the device in any particular case, e.g., whether device caliber is appropriate for certain procedure.

Head portion 116 may further include contact sensors 177, 179 connected to CPU to provide indication as to when tissue interposed between anvil assembly 130 and shell assembly 131 is initially brought into contact with tissue contacting surface 130a. Thus, a substantially uncompressed thickness of tissue may be measured by monitoring magnetic field sensor 160 when tissue is initially brought into contact with tissue contacting surface 130a.

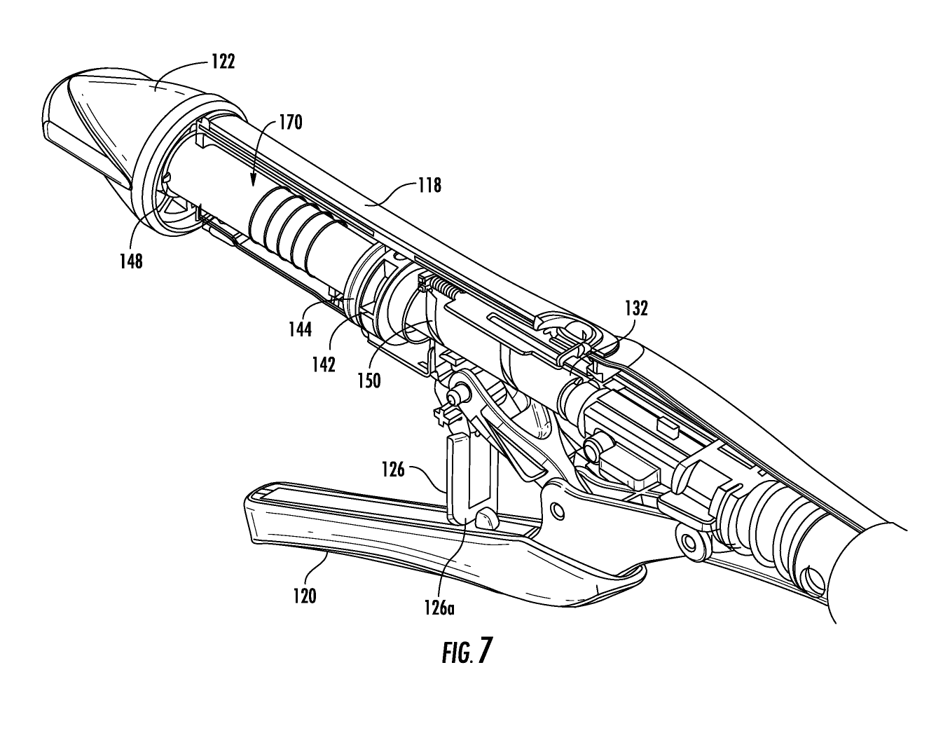

With reference now to FIGS. 7 and 8, the approximation mechanism includes approximation knob 122, a drive screw 132, a rotatable sleeve 170, and an anvil retainer 138 (FIG. 8) for supporting an anvil assembly 130. Rotatable sleeve 170 includes a substantially cylindrical hollow body portion and a substantially cylindrical collar 142 which together define a central bore. Collar 142 has an annular groove 144 formed thereabout, which is dimensioned to receive an inwardly extending flange formed on an inner wall of handle assembly 118. Engagement between groove 144 and the flanges axially fixes sleeve 170 within handle assembly 118 while permitting rotation of sleeve 170 in relation to handle assembly 118. A pair of diametrically opposed elongated ribs 148 is positioned or formed on the outer surface of the body portion. Approximation knob 122 includes a pair of internal slots (not shown) positioned to receive ribs 148 of sleeve 170 to rotatably fix sleeve 170 to knob 122, such that rotation of knob 122 causes concomitant rotation of sleeve 170.

The proximal half of screw 132 includes a helical channel 150 and is dimensioned to be slidably positioned within the central bore of rotatable sleeve 170. Since sleeve 170 is axially fixed with respect to handle assembly 118, rotation of sleeve 170 about screw 132 causes a pin (not shown) to move along channel 150 of screw 132 to effect axial movement of screw 132 within handle assembly 118.

In use, when approximation knob 122 is manually rotated, rotatable sleeve 170 is rotated about the proximal end of screw 132 to move a pin along helical channel 150 of screw 132. Since sleeve 170 is axially fixed to handle assembly 118, as the pin is moved through channel 150, screw 132 is advanced or retracted within handle assembly 118. As a result, top and bottom screw extensions (not shown), which are fastened to the distal end of screw 132 and to anvil retainer 138, are moved axially within elongated body portion 114. Since anvil assembly 130 is secured to the distal end of anvil retainer 138, rotation of approximation knob 122 will effect movement of anvil assembly 130 in relation to shell assembly 131 between spaced and approximated positions.

With shell assembly 131 and anvil assembly 130 in spaced relation to one another, target tissue is placed therebetween. With the target tissue positioned between shell assembly 131 and anvil assembly 130, anvil assembly 130 is approximated towards shell assembly 131 until the target tissue makes a contact with contact sensors 177, 179. At this time, magnetic field sensors 160 may measure the magnetic field and send the data to CPU, which determines the thickness of substantially uncompressed tissue. The tissue thickness in the uncompressed state is displayed and/or recorded. Thereafter, shell assembly 131 and anvil assembly 130 are further approximated until a desired gap between shell assembly 131 and anvil assembly 130 is obtained. A compressed tissue thickness may be measured by magnetic field sensors 160, during or after approximation of shell assembly 131 and anvil assembly 130.

In operation, following purse string suturing of a first tissue “T1” to anvil assembly 130 and purse string suturing of a second tissue “T2” to shell assembly 131 (FIG. 8), approximation knob 122 is rotated to approximate anvil assembly 130 towards shell assembly 131. As anvil assembly 130 and shell assembly 131 are approximated toward one another, first and second tissue “T1, T2” are extended toward one another and are tensioned. As first and second tissue “T1, T2” are tensioned, first and second tissue “T1, T2” tend to constrict around anvil assembly 130 and shell assembly 131, respectively. This constriction exerts a force on each respective force measuring sensor 164, 166. The force measured by each force measuring sensor 164, 166 may be converted, using known algorithms, to a value of tension force which is being exerted on each tissue “T1, T2”. Surgical instrument 100 may include a gauge 140 (FIG. 6) supported on stationary handle 118 of handle assembly 112. Each sensor 160 may be operatively connected to gauge 140. Gauge 140 functions to display, in real time, selected operational parameters, such as, for example, tissue contact, tissue compression, tissue tension, etc.

During a surgical anastomotic procedure, the tension on first and second tissues “T1, T2” may be monitored to maintain the tension exerted thereon at or below a predetermined threshold level. For example, if the tension exerted on each tissue “T1, T2”, either alone or in combination, exceeds a predetermined threshold level, elevated tension acts on the staple line and may result in undue strains exerted on the staples and/or the staple line.

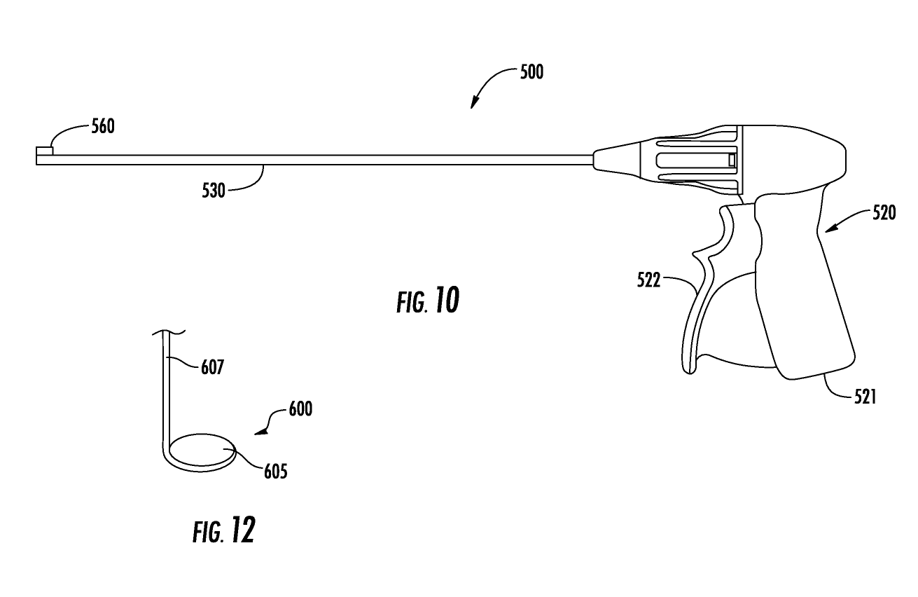

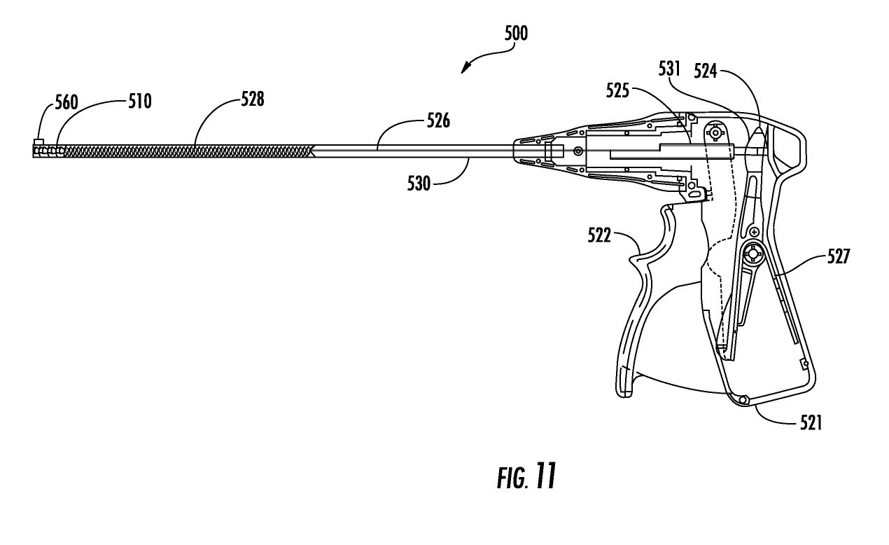

With reference now to FIGS. 10 and 11, a surgical instrument including a magnetic field sensor 560 in accordance with an embodiment of the present disclosure is generally designated as 500. Surgical instrument 500 is configured to serially deploy at least one surgical anchor 510 to attach a prosthesis in place in the repair of a defect in tissue such as an inguinal hernia. Surgical instrument 500 includes a handle assembly 520 and a delivery tube 530 extending distally from handle assembly 520. Handle assembly 520 includes a stationary handle 521 and a firing trigger 522. Reference may be made to U.S. Pat. No. 7,758,612, the entire contents of which are incorporated herein by reference, for a more detailed discussion of the structure and operation of surgical instrument 500 and surgical anchor 510.

With particular reference now to FIGS. 10 and 12, magnetic field sensor 560 is disposed at a distal portion of delivery tube 530. The placement of magnetic field sensor 560 at, e.g., a distal-most portion of delivery tube 530, facilitates use thereof in conjunction with a magnet assembly 600. Magnet assembly 600 includes a magnet 605 and an elongated support 607 extending from magnet 605. Magnet 605 and magnetic field sensor 560 may be constructed as described above in connection with embodiments of FIGS. 1-9.

Magnet 605 may be positioned on one side of tissue to be measured and magnetic field sensor 560 may be placed on an opposing side of tissue. Magnetic field sensor 560 generates a detectable signal in response to a magnetic field of magnet 605. Magnetic field sensor 560 may be connected to a processor (not shown). The processor may calculate the distance between magnet 605 and magnetic field sensor 560, i.e., thickness of tissue, based on the detectable signal.

With respect to FIG. 11, upon determining thickness of tissue, the surgeon may then apply surgical anchor 510 to tissue by pulling trigger 522 toward stationary handle 521. When the surgeon pulls trigger 522 toward stationary handle 521, a lever 524 rotates counterclockwise such that a cam surface 531 of lever 524 contacts a piston 525 which drives an anchor carrier rod 526 distally. A torsion spring 527 is compressed as lever 524 is rotated counterclockwise. Anchor carrier 526 is urged distally within a queuing spring 528, which, in turn, urges the distal-most anchor 510 past a distal end of delivery tube 530. In this manner, anchor 510 penetrates through the prosthesis and tissue.

Although the illustrative embodiments of the present disclosure have been described herein with reference to the accompanying drawings, the above description, disclosure, and figures should not be construed as limiting, but merely as exemplifications of particular embodiments. For example, in the embodiments described in connection with FIGS. 1 to 5, it is envisioned that the plurality of magnets 362a-d may be disposed on tissue contacting surface 324a of anvil assembly 324, and the plurality of magnetic field sensors 360a-d may be disposed on tissue contacting surface 322a of cartridge assembly 322. Likewise, with respect to the embodiments described in connection with FIGS. 6 to 9, it is envisioned that the plurality of magnets 162 may be disposed on tissue contacting surface 130a of anvil assembly 130, and the plurality of magnetic field sensors 160 may be disposed on tissue contacting surface 131a of shell assembly 131. In addition, it is envisioned that magnet 605 may be placed on a distal portion of delivery tube 530, and magnetic field sensor 560 may be provided as a separate element from surgical instrument 500. It is to be understood, therefore, that the disclosure is not limited to those precise embodiments, and that various other changes and modifications may be effected therein by one skilled in the art without departing from the scope or spirit of the disclosure.

What is claimed is:

1. A surgical instrument comprising:
an end effector including first and second tissue contacting surfaces configured to receive tissue therebetween, the first tissue contacting surface movable relative to the second tissue contacting surface between a spaced apart position and an approximated position; and
a magnetic field sensor assembly including a first magnetic field sensor disposed on the first tissue contacting surface and a first magnet disposed on the second tissue contacting surface; and
a processor connected to the first magnetic field sensor, wherein the processor determines a distance between the first and second tissue contacting surfaces based on a detectable signal received from the first magnetic field sensor.

2. The surgical instrument according to claim 1, further including a contact sensor disposed on the first tissue contacting surface, the contact sensor monitoring contact between tissue and the first tissue contacting surface.

3. The surgical instrument according to claim 1, wherein the first tissue contacting surface is pivotably coupled with the second tissue contacting surface about a pivot.

4. The surgical instrument according to claim 3, wherein the first magnetic field sensor is disposed adjacent the pivot.

5. The surgical instrument according to claim 4, wherein the magnetic field sensor assembly further includes a second magnetic field sensor disposed distal of the first magnetic field sensor and a second magnet disposed distal of the first magnet, such that during approximation of the first tissue contacting surface toward the second tissue contacting surface, the first magnetic field sensor contacts tissue while the second magnetic field sensor is spaced apart from tissue.

6. The surgical instrument according to claim 1, wherein the first magnet and the first magnetic field sensor are in a superposed relation in the approximated position.

7. The surgical instrument according to claim 1, wherein the first magnetic field sensor is a Hall effect sensor.

8. The surgical instrument according to claim 1, wherein the first magnetic field sensor comprises a magnetoresistive film.

9. A method of determining tissue thickness, the method comprising:
placing tissue between a first tissue contacting surface and a second tissue contacting surface of an end effector of a surgical instrument;
approximating the first and second tissue contacting surfaces;
generating a detectable signal, the detectable signal generated by a magnetic field sensor on the first tissue contacting surface in response to a magnetic field of a magnet on the second tissue contacting surface; and
calculating a distance between the first and second tissue contacting surfaces based on the detectable signal.

10. The method according to claim 9, further comprising determining an initial contact between tissue and the first tissue contacting surface.

11. The method according to claim 10, wherein generating a detectable signal includes generating the detectable signal at the time of initial contact between tissue and the first tissue contacting surface.

12. A method of determining tissue thickness, the method comprising:
placing a magnet on a first side of tissue;
placing a magnetic field sensor mounted on a surgical instrument on a second side of tissue, the second side opposite of the first side;
generating a detectable signal, the detectable signal generated by the magnetic field sensor in response to a magnetic field of the magnet; and
calculating a distance between the magnet and the magnetic field sensor based on the detectable signal.
